(19) United States
(12) Patent Application Publication
(10) Pub. No.: US 2026/0216399 A1
(43) Pub. Date: Jul. 30, 2026

(54) Polymer Composition for Medical Implants and Implants Made Therefrom

(71) Applicant: Celanese International Corporation, Irving, TX (US)

(72) Inventors: Victoria Black, Cincinnati, OH (US); Rainer Walkenhorst, Melle (DE)

(21) Appl. No.: 19/454,494

(22) Filed: Jan. 21, 2026

Related U.S. Application Data
(60) Provisional application No. 63/750,807, filed on Jan. 29, 2025.

(51) Int. Cl. A61L 27/16; A61F 2/34; A61F 2/38

(52) U.S. Cl. CPC A61L 27/16; A61F 2/34; A61F 2/389; A61L 2430/02; A61L 2430/24

(57) ABSTRACT

A biocompatible polymer composition is disclosed comprised of high density polyethylene particles. The high density polyethylene particles can comprise an ultrahigh molecular weight polyethylene polymer. The particles can display a relatively high bulk density. In addition, the polyethylene polymer can be made with controlled amounts of aluminum, titanium, and other non-polymer components.

### CROSS REFERENCE TO RELATED APPLICATIONS

This application claims priority to the benefit of U.S. Provisional Application No. 63/750,807, filed Jan. 29, 2025, which is expressly incorporated herein by reference in its entirety.

### BACKGROUND

Polyethylene has become established as an exceptionally useful engineering material in a variety of applications, in part because of its unique combination of desirable properties. For instance, high molecular weight polyethylene polymer particles may exhibit an improved abrasion resistance, chemical resistance, lubricity, impact strength, stress crack resistance, heat deflection temperature, wear resistance, and energy absorption capacity at high stress rates in comparison to other thermoplastic polymers.

High molecular weight polyethylene polymers generally include linear polyethylene polymers and can be differentiated from other polyethylene grades by a very high degree of polymerization. In fact, the high degree of polymerization is responsible for the fact that these polymers do not flow well when melted and, in fact, can display a melt flow index of 0 grams per 10 mins. Therefore, special processing methods like pressure sintering and ram extrusion are used to form these polymers into articles.

High molecular weight polyethylene polymers as described above, however, have various superior properties that make them desirable for a number of applications. For example, high molecular weight polyethylene polymers can display excellent mechanical properties. High molecular weight polyethylene polymers also have a relatively broad operating temperature range and are extremely wear resistant. In addition, high molecular weight polyethylene has great abrasion resistant properties which can be as good or better than steel.

High molecular weight polyethylene polymers can also be produced to be biocompatible. Due to their mechanical and physical properties, high molecular weight polyethylene polymers are well suited for use in producing surgical implants. The ability to use high molecular weight polyethylene polymers to produce surgical implants, however, is highly regulated. For instance, ASTM F648-21 provides the standards under which ultrahigh molecular weight polyethylene powders can be used for medical devices, such as implants. ASTM F648-21, which is incorporated herein by reference, specifies the chemical composition, physical properties, and performance characteristics that are required in order to use an ultrahigh molecular weight polyethylene in medical implant applications. ASTM F648 categorizes ultrahigh molecular weight polyethylene into different types, particularly Type 1, Type 2, and Type 3, which are distinguished based on how the material is processed, such as whether the polymer is virgin, irradiated, remelted, or annealed. The standard also covers the specific methods by which ultrahigh molecular weight polyethylenes should be processed to ensure that the material meets the stringent requirements necessary for use in the human body. This includes processes like compression molding, radiation cross-linking, and post-irradiation treatments. The required properties of ultrahigh molecular weight polyethylenes for use in medical applications under ASTM F648 include requirements related to density, molecular weight, tensile strength, elongation, and impact resistance. These properties are intended to ensure that the material is suitable for long-term implantation in the human body, especially when used to produce joint prostheses, such as acetabular liners, hip prosthetics, tibial inserts (tibial plateau), knee replacements, and other similar orthopedic devices.

Type 1 ultrahigh molecular weight polyethylene polymers under ASTM F648 lists the requirements for using virgin polymers in medical applications. In the past, very few commercially available polymers have satisfied these stringent specifications. However, Celanese International, Inc. was able to develop an ultrahigh molecular weight polyethylene grade (GUR 1020) that met all of the requirements of ASTM F648. Consequently, this particular grade of high molecular weight polyethylene has demonstrated great success in producing medical devices for implantation into the human body.

Ultrahigh molecular weight polyethylene polymers that have met the requirements of ASTM F648 (Type 1) in the past have displayed a relatively low bulk density. A low bulk density is desirable and preferred in many applications. Higher bulk density polymers, however, may provide benefits in certain situations. For example, a higher bulk density powder can lead to efficiencies during the process of compression molding or ram extrusion. In the past, however, problems have been experienced in producing an ultrahigh molecular weight polyethylene polymer having a high bulk density that also meets all of the requirements of ASTM F648. For instance, such polymers produced in the past do not display the purity requirements and/or impact resistant requirements of the ASTM standard. In view of the above, a need exists for an ultrahigh molecular weight polyethylene polymer that has a relatively high bulk density and meets all of the requirements of ASTM F648 related to virgin polymers.

### SUMMARY

The present disclosure is generally directed to a high density and high molecular weight polyethylene polymer that is well suited for medical applications. The high molecular weight polyethylene polymer, for instance, can be formulated to meet all of the requirements of ASTM Standard F648 (2021), Type 1. Consequently, the high molecular weight polyethylene polymer can be used to produce surgical implants for implantation into the human body.

In one embodiment, for instance, the present disclosure is directed to a polymer composition for producing implants. The polymer composition comprises an ultrahigh molecular weight polyethylene in the form of particles. The ultrahigh molecular weight polyethylene has an average molecular weight of from about 3,000,000 g/mol to about 12,000,000 g/mol. The ultrahigh molecular weight polyethylene particles have an average particle size (D50) of from about 100 microns to about 200 microns, the polymer composition displays an Izod impact strength of at least about 126 kJ/m² when tested according to ASTM F 648-21, Annex A1. The polymer composition contains the ultrahigh molecular weight polyethylene in an amount of about 99% by weight or greater. The ultrahigh molecular weight polyethylene has a density of from about 927 kg/m³ to about 944 kg/m³ when tested according to ASTM D792 (2020) or D1505 (2018). The ultrahigh molecular weight polyethylene particles display a bulk density of greater than about 0.46 g/cm³, such as greater than about 0.48 g/cm³, such as greater than about 0.49 g/cm³, such as greater than about 0.5 g/cm³, such as greater than about 0.51 g/cm³, and less than about 0.6 g/cm³.

The ultrahigh molecular weight polyethylene can display high purity properties. For instance, the ultrahigh molecular weight polyethylene can contain aluminum in an amount from about 10 ppm to about 50 ppm, such as in an amount from about 10 ppm to about 35 ppm, such as in an amount from about 10 ppm to about 20 ppm. The ultrahigh molecular weight polyethylene can contain titanium in an amount less than about 15 ppm, such as less than about 12 ppm, such as less than about 10 ppm. The polymer composition can be formulated to be free of stabilizers, antioxidants, and processing aids. The ultrahigh molecular weight polyethylene can comprise a polyethylene homopolymer according to ASTM D4020. In one aspect, the polymer composition contains the ultrahigh molecular weight polyethylene polymer in an amount greater than about 99.9% by weight. The ultrahigh molecular weight polyethylene polymer can contain calcium in an amount less than about 5 mg/kg or less. The ultrahigh molecular weight polyethylene polymer can also contain chlorine in an amount of about 30 mg/kg or less.

In one aspect, the ultrahigh molecular weight polyethylene particles have an average particle size of from about 120 microns to about 160 microns. The ultrahigh molecular weight polyethylene can display an average molecular weight of from about 3,800,000 g/mol to about 4,800,000 g/mol. The ultrahigh molecular weight polyethylene can display a viscosity number of from about 2,000 ml/g to about 3,200 ml/g when tested according to ASTM D4020 (2018).

The present disclosure is also directed to a polymer composition for producing implants that contains an ultrahigh molecular weight polyethylene in the form of particles and having a controlled amount of metals. The ultrahigh molecular weight polyethylene can have an average molecular weight of from about 3,000,000 g/mol to about 8,000,000 g/mol and can have an average particle size of from about 100 microns to about 200 microns. The polymer composition can display an Izod impact strength of at least about 126 kJ/m² when tested according to ASTM F648-21, Annex A1. The polymer composition can contain the ultrahigh molecular weight polyethylene in an amount of about 99% by weight or greater. The ultrahigh molecular weight polyethylene can have a density of from about 927 kg/m³ to about 944 kg/m³. In accordance with the present disclosure, the ultrahigh molecular weight polyethylene can contain aluminum in an amount from about 10 ppm to about 50 ppm, such as from about 10 ppm to about 35 ppm. In one aspect, the ultrahigh molecular weight polyethylene can also contain titanium in an amount less than about 15 ppm, such as less than about 12 ppm, such as less than about 10 ppm.

Polymer compositions as described above are particularly well suited for producing implants. In one aspect, the implants can be produced through compression molding or ram extrusion.

Implants that can be made according to the present disclosure include a hip prothesis, an acetabular cup, a tibial plateau, a knee prothesis, or the like.

Other features and aspects of the present disclosure are discussed in greater detail below.

### BRIEF DESCRIPTION OF THE DRAWINGS

A full and enabling disclosure of the present disclosure is set forth more particularly in the remainder of the specification, including reference to the accompanying figure, in which:

FIG. 1 is a cross-sectional view of medical implants that can be made in accordance with the present disclosure.

Repeat use of reference characters in the present specification and drawings is intended to represent the same or analogous features or elements of the present invention.

### DEFINITIONS

The melt flow rate of a polymer or polymer composition is measured according to ISO 1133 at 190° C. and at a load of 21.6 kg.

Particle size including average particle size (d50) and particle size distribution is measured using laser diffraction/light scattering according to ISO 13320(2020).

The average molecular weight of a polymer is determined using the Margolies'equation. Molecular weight can be determined by first measuring the viscosity number according to DIN EN ISO 1628-3 (2010). Dry powder flow is measured using a 25 mm nozzle.

Tensile modulus, tensile stress at yield, tensile strain at yield, tensile stress at 50% break, tensile stress at break, and tensile nominal strain at break are all measured according to ISO 527-2/1B (2012).

As used herein, bulk density is measured according to ISO 60 (2023) (formerly DIN 53466).

The amount of titanium, aluminum, calcium, and chlorine contained in a polyethylene polymer is determined according to ASTM F648-21.

Polymer density is determined according to ASTM D792 or D1505.

Izod impact strength is measured according to ASTM F648-21, Annex A1.

### DETAILED DESCRIPTION

It is to be understood by one of ordinary skill in the art that the present discussion is a description of exemplary embodiments only and is not intended as limiting the broader aspects of the present disclosure.

In general, the present disclosure is directed to a biocompatible polymer composition well suited to producing medical implants. The polymer composition, for instance, can contain a high density polyethylene polymer, such as an ultrahigh molecular weight polyethylene polymer, that meets all of the requirements of ASTM F648-21, which is the standard specification for fabricating surgical implants. In one aspect, the high density polyethylene polymer particles made in accordance with the present disclosure display a relatively high bulk density. The high density polyethylene polymer can also be produced with controlled amounts of impurities from the Ziegler-Natta catalyst. For instance, the high density polyethylene polymer can contain controlled amounts of aluminum, titanium, and other elements.

In the past, high density polyethylene polymers were produced that met the requirements of ASTM F648, Type 1 for medical implants. The commercially available high density polyethylene polymer particles have demonstrated excellent properties in producing implants. The high density polyethylene polymer particles, however, have a relatively low bulk density which can be advantageous in various applications. In other applications, however, high density polyethylene particles having a higher bulk density may provide some advantages and benefits. For instance, high density polyethylene particles having a relatively high bulk density may offer some processing advantages when being compression molded or ram extruded into implants. In the past, however, problems were experienced in producing high density polyethylene particles that displayed a relatively high bulk density while still possessing the mechanical properties, particularly impact strength, that is required under ASTM F648. The high density polyethylene particles of the present disclosure address the above problems experienced in the past providing for a polymer that not only has relatively high impact strength when tested under ASTM F648 in combination with a relatively high bulk density.

The high density polyethylene polymer composition can contain particles having an average particle size (d50) of greater than about 50 microns, such as greater than about 100 microns, such as greater than about 120 microns, such as greater than about 140 microns, such as greater than about 160 microns, such as greater than about 180 microns, and less than about 220 microns, such as less than about 200 microns, such as less than about 180 microns, such as less than about 160 microns, such as less than about 140 microns.

The high density polyethylene polymer particles as described above can have a relatively high bulk density. Bulk density can be measured according to ISO 60. In one aspect, the bulk density of the high density polyethylene polymer particles is greater than about 0.46 g/cm³, such as greater than about 0.47 g/cm³, such as greater than about 0.48 g/cm³, such as greater than about 0.49 g/cm³, such as greater than about 0.5 g/cm³, and less than about 0.6 g/cm³, such as less than about 0.55 g/cm³, such as less than about 0.53 g/cm³.

The high density polyethylene polymer used to form the polymer composition of the present disclosure can generally have a density of about 0.92 g/cm³ or greater, such as about 0.93 g/cm³ or greater, such as about 0.94 g/cm³ or greater, and generally less than about 1 g/cm³, such as less than about 0.97 g/cm³.

The high density polyethylene polymer can be made from over 90% ethylene derived units, such as greater than 95% ethylene derived units, or from 100% ethylene derived units. The polyethylene can be a homopolymer or a copolymer, including a terpolymer, having other monomeric units. In one aspect, the high density polyethylene can be a high density polyethylene homopolymer according to the specification of ASTM D4020 (2018).

The high density polyethylene can be a very high molecular weight polyethylene and/or an ultrahigh molecular weight polyethylene.

“Very-high molecular weight polyethylene” refers to polyethylene compositions with a weight average molecular weight of less than about 3×10⁶ g/mol and more than about 1×10⁶ g/mol. In some embodiments, the molecular weight of the very-high molecular weight polyethylene composition is between about 2×10⁶ g/mol and less than about 3×10⁶ g/mol.

“Ultra-high molecular weight polyethylene” refers to polyethylene compositions with an average molecular weight of at least about 3×10⁶ g/mol. In some embodiments, the molecular weight of the ultra-high molecular weight polyethylene composition is between about 3×10⁶ g/mol and about 30×10⁶ g/mol, or between about 3×10⁶ g/mol and about 20×10⁶ g/mol, or between about 3×10⁶ g/mol and about 10×10⁶ g/mol, or between about 3×10⁶ g/mol and about 6×10⁶ g/mol.

In one embodiment, the high density polyethylene polymer comprises an ultrahigh molecular weight polyethylene polymer. For instance, the high density polyethylene polymer can have an average molecular weight of greater than about 1,000,000 g/mol, such as greater than about 3,000,000 g/mol, such as greater than about 4,000,000 g/mol, such as greater than about 4,500,000 g/mol, such as greater than about 5,000,000 g/mol, such as greater than about 5,500,000 g/mol, such as greater than about 6,000,000 g/mol, such as greater than about 6,500,000 g/mol. The molecular weight of the high density polyethylene polymer is generally less than about 12,000,000 g/mol, such as less than about 9,000,000 g/mol, such as less than about 8,000,000 g/mol, such as less than about 7,000,000 g/mol, such as less than about 6,000,000 g/mol, such as less than about 5,500,000 g/mol, such as less than about 5,000,000 g/mol.

Any method known in the art can be utilized to synthesize the high density polyethylene polymer particles. The polyethylene powder is typically produced by the catalytic polymerization of ethylene monomer or optionally with one or more other 1-olefin co-monomers, the 1-olefin content in the final polymer being less or equal to 10% of the ethylene content, with a heterogeneous catalyst and an organo aluminum or magnesium compound as cocatalyst. The ethylene can be polymerized in gaseous phase or slurry phase at relatively low temperatures and pressures. The polymerization reaction may be carried out at a temperature of between 50° C. and 100° C. and pressures in the range of 0.02 and 2 MPa.

The molecular weight of the polyethylene can be adjusted by adding hydrogen. Altering the temperature and/or the type and concentration of the co-catalyst may also be used to fine tune the molecular weight.

Suitable catalyst systems include but are not limited to Ziegler-Natta type catalysts, metallocene catalysts, and/or post metallocene catalysts. Typically Ziegler-Natta type catalysts are derived by a combination of transition metal compounds of Groups 4 to 8 of the Periodic Table and alkyl or hydride derivatives of metals from Groups 1 to 3 of the Periodic Table. Transition metal derivatives used usually comprise the metal halides or esters or combinations thereof. Exemplary Ziegler-Natta catalysts include those based on the reaction products of organo aluminum or magnesium compounds, such as for example but not limited to aluminum or magnesium alkyls and titanium, vanadium or chromium halides or esters. The heterogeneous catalyst might be either unsupported or supported on porous fine grained materials, such as silica or magnesium chloride. Such support can be added during synthesis of the catalyst or may be obtained as a chemical reaction product of the catalyst synthesis itself.

In one embodiment, a suitable catalyst system could be obtained by the reaction of a titanium(IV) compound with a trialkyl aluminum compound in an inert organic solvent at temperatures in the range of −40° C. to 100° C., preferably −20° C. to 50° C. The concentrations of the starting materials are in the range of 0.1 to 9 mol/L, preferably 0.2 to 5 mol/L, for the titanium(IV) compound and in the range of 0.01 to 1 mol/L, preferably 0.02 to 0.2 mol/L for the trialkyl aluminum compound. The molar ratio of titanium and aluminum in the final mixture can be in the range of 1:0.01 to 1:4.

In another embodiment, a suitable catalyst system is obtained by a one or two-step reaction of a titanium(IV) compound with a trialkyl aluminum compound in an inert organic solvent at temperatures in the range of −40° C. to 200° C., preferably −20° C. to 150° C. In the first step, the titanium(IV) compound is reacted with the trialkyl aluminum compound at temperatures in the range of −40° C. to 100° C., preferably −20° C. to 50° C. using a molar ratio of titanium to aluminum in the range of 1:0.1 to 1:0.8. The concentrations of the starting materials are in the range of 0.1 to 9.1 mol/L, preferably 5 to 9.1 mol/L, for the titanium(IV) compound and in the range of 0.05 and 1 mol/L, preferably 0.1 to 0.9 mol/L for the trialkyl aluminum compound. The titanium component is added to the aluminum compound over a period of 0.1 min to 800 min, preferably 30 min to 600 min. In a second step, if applied, the reaction product obtained in the first step is treated with a trialkyl aluminum compound at temperatures in the range of −10° C. to 150° C., preferably 10° C. to 130° C. using a molar ratio of titanium to aluminum in the range of 1:0.01 to 1:5.

In yet another embodiment, a suitable catalyst system is obtained by a procedure wherein, in a first reaction stage, a magnesium alcoholate is reacted with a titanium chloride in an inert hydrocarbon at a temperature of 50° to 100° C. In a second reaction stage, the reaction mixture formed is subjected to heat treatment for a period of about 10 to 100 hours at a temperature of 110° to 200° C. accompanied by evolution of alkyl chloride until no further alkyl chloride is evolved, and the solid is then freed from soluble reaction products by washing several times with a hydrocarbon.

In a further embodiment, catalysts supported on silica, such as for example the commercially available catalyst system Sylopol 5917 can also be used.

In one embodiment, especially when producing a relatively high molecular weight polyethylene, a metallocene-type catalyst may be used. For example, in one embodiment, two different metallocene-type catalysts can be used to produce the polyethylene polymer. For instance, the metallocene catalysts may be made from metals, such as hafnium and/or chromium.

Using such catalyst systems, the polymerization is normally carried out in suspension at low pressure and temperature in one or multiple steps, continuous or batch. The polymerization temperature is typically in the range of 30° C. to 130° C., preferably in the range of 50° C. and 90° C. and the ethylene partial pressure is typically less than 10 MPa, preferably 0.05 and 5 MPa. Aluminum compounds can be used as co-catalyst such that the ratio of Al:Ti (co-catalyst versus catalyst) is in the range of 0.01 to 100:1, more preferably in the range of 0.03 to 50:1. The solvent is an inert organic solvent as typically used for Ziegler type polymerizations. Examples are butane, pentane, hexane, cyclohexane, octane, nonane, decane, their isomers and mixtures thereof. The polymer molecular mass is controlled through feeding hydrogen. The ratio of hydrogen partial pressure to ethylene partial pressure is in the range of 0 to 50, preferably the range of 0 to 10. The polymer is isolated and dried. Salts of long chain fatty acids may be added as a stabilizer.

Generally a cocatalyst such as alumoxane or alkyl aluminum or alkyl magnesium compound is also employed. Other suitable catalyst systems include Group 4 metal complexes of phenolate ether ligands.

In one aspect, the high density polyethylene particles are produced in a manner that makes the polymer well suited for medical applications, such as for producing implants and prosthetics. For instance, in one embodiment, the high density polyethylene polymer is made such that the polymer conforms with the requirements of ASTM F648 (2021). For instance, the high density polyethylene polymer can be produced in a manner that contains a very low amount of impurities. For instance, the high density polyethylene polymer can contain titanium in an amount less than about 40 ppm, such as in an amount less than about 20 ppm, such as in an amount less than about 15 ppm, such as in an amount less than about 12 ppm, such as in an amount less than about 10 ppm. The high density polyethylene polymer can contain aluminum in an amount less than about 50 ppm, such as in an amount less than about 40 ppm, such as in an amount less than about 30 ppm, such as in an amount less than about 20 ppm, such as in an amount less than about 15 ppm, such as in an amount less than about 13 ppm, and in an amount greater than about 1 ppm, such as greater than about 5 ppm, such as greater than about 6 ppm, such as greater than about 7 ppm. In addition, the high density polyethylene polymer can contain calcium in an amount less than about 50 ppm, such as in an amount less than about 5 ppm, and can contain chlorine in an amount less than about 90 ppm, such as in an amount less than about 30 ppm. Titanium, aluminum, calcium, and chlorine content can be determined according to ASTM F648.

The high density polyethylene polymer, in one embodiment, can display a viscosity number when tested according to ASTM D4020 (0.02%) of greater than about 2,000 mL/g, such as greater than about 2,200 mL/g, such as greater than about 2,400 mL/g, such as greater than about 2,600 mL/g. In one aspect, the high density polyethylene polymer can display a viscosity number of from about 2,000 mL/g to about 3,200 mL/g. In an alternative embodiment, the high density polyethylene polymer can display a viscosity number of greater than about 3,200 mL/g and less than about 10,000 mL/g, such as less than about 6,000 mL/g, such as less than about 5,000 mL/g.

The high density polyethylene polymer can also display excellent mechanical properties. For instance, when tested at 23° C. according to ASTM D638 (2022), Type IV, 1.5 mm±0.5 mm and at a rate of 5.08 cm/min, the polymer composition can display an ultimate tensile strength of greater than about 10 MPa, such as greater than about 20 MPa, such as greater than about 27 MPa, such as greater than about 35 MPa, such as greater than about 40 MPa, and less than about 100 MPa, and can display a yield of greater than about 19 MPa, such as greater than about 21 MPa, and less than about 35 MPa.

The high density polyethylene polymer can also display excellent Izod impact strength properties. For instance, when tested according to Izod impact strength according to ASTM F648-21, Annex A1, the high density polyethylene polymer composition can display an impact strength of greater than about 25 kJ/m², such as greater than about 73 kJ/m², such as greater than about 126 kJ/m², and less than about 200 kJ/m². As described above, the above impact strength properties can be achieved according to the present disclosure with a high density polyethylene polymer having a relatively high bulk density and that meets all the other requirements of F648, Type 1.

When tested according to ASTM D638 as described above, the high density polyethylene polymer composition can display an elongation of greater than about 250%, such as greater than about 340%, such as greater than about 380%, and less than about 600%.

When the polymer composition of the present disclosure is formulated to produce implants, the polymer composition, in one aspect, contains virtually no other components or ingredients except for the high density polyethylene polymer particles. For instance, the polymer composition can contain the high density polyethylene polymer in an amount greater than about 99% by weight, such as in an amount greater than about 99.3% by weight, such as in an amount greater than about 99.5% by weight, such as in an amount greater than about 99.8% by weight, such as in an amount greater than about 99.9% by weight. The polymer composition, for instance, can be free of stabilizers, processing aids, or antioxidants.

The high density polyethylene polymer generally has a relatively low melt flow rate. The melt flow rate, for instance, is generally less than about 3 g/10 min at 190° C. and at a load of 21.6 kg according to ISO 1133 (2022). For instance, the melt flow rate of the high density polyethylene polymer can be less than about 2.8 g/10 min, such as less than about 2.5 g/10 min, such as less than about 2.2 g/10 min, such as less than about 2 g/10 min, and generally about 0 g/10 min or greater.

The high density polyethylene polymer composition of the present disclosure can be subjected to various different processes and techniques in order to form molded articles. In one embodiment, molded articles, such as implants, can be formed without crosslinking the high density polyethylene polymer. In an alternative embodiment, the high density polyethylene polymer can be crosslinked.

The high density polyethylene polymer can be crosslinked using any suitable method. For instance, a chemical crosslinking agent can be incorporated into the polymer or the polymer particles can be exposed to irradiation. For example, the high density polyethylene particles can be crosslinked using either x-rays, e-beam, or gamma rays through a process called radiation crosslinking. This process involves exposing the polymer to high-energy radiation which causes the formation of free radicals within the polymer chains. These free radicals then initiate crosslinking reactions leading to the formation of a three-dimensional network within the polymer particles.

For example, in one embodiment, the high density polyethylene particles can be exposed to either x-rays or gamma rays from a radiation source. Both x-rays and gamma rays are forms of ionizing radiation that have sufficient energy to penetrate the material and induce chemical reactions within the polymer chains. When the radiation interacts with the polymer, it generates the free radicals within the polymer chains by breaking chemical bonds. These free radicals are highly reactive species with unpaired electrons.

The free radicals produced during radiation exposure initiate crosslinking reactions between neighboring polymer chains. This process involves the formation of covalent bonds between polymer molecules, leading to the creation of a three-dimensional network structure. After the desired level of crosslinking is achieved, optionally, the polymer particles may undergo post-treatment steps such as cooling or annealing.

The amount of radiation used to crosslink the high density polyethylene can vary. When using X-ray radiation or gamma radiation, for example, the polymer particles can be exposed to greater than about 100 kGy, such as greater than about 150 kGy, such as greater than about 200 kGy, such as greater than about 250 kGy, and less than about 1,000 kGy, such as less than about 500 kGy. When using gamma radiation, the polymer particles can be exposed to greater than about 100 kGy, such as greater than about 150 kGy, such as greater than about 200 kGy, such as greater than about 250 kGy, and less than about 1,000 kGy, such as less than about 500 kGy, such as less than about 300 kGy.

Whether the high density polyethylene polymer is crosslinked or remains uncrosslinked, the polymer composition of the present disclosure can be molded into articles using various methods. For instance, the high density polyethylene particles can be molded into an article using compression molding, ram extrusion, or hot isostatic pressing. Compression molding can include direct compression molding which is also referred to net shape compression molding. It is believed that the high density polyethylene polymer particles of the present disclosure having a relatively high bulk density may provide various processing efficiencies when using one or more of the above methods.

In compression molding, the polymer particles are placed into a heated mold cavity where heat and pressure are applied to consolidate the particles into the shape of the mold. The process typically allows for precise control of the final article's dimensions and surface finish, and is particularly suitable for producing complex or net-shape parts. In contrast, ram extrusion involves forcing the polymer particles through a heated die using a plunger or ram, producing a continuous profile with a uniform cross-section. The profile can then be cut, machined, or carved to form the final article. Ram extrusion generally enables faster production of long or linear parts and can be advantageous for creating articles with consistent mechanical properties along the extruded length. The choice between compression molding and ram extrusion may depend on factors such as the desired part geometry, production speed, and dimensional tolerances, and the high bulk density of the high density polyethylene polymer particles of the present disclosure can provide benefits in both processes by improving material handling and reducing cycle times.

Once the implant is molded, the article can optionally be subjected to various post treatments if desired. For instance, in one embodiment, the molded article can be subjected to heat treatment which may increase the strength of the article. The surface of the molded article can also optionally be subjected to a treatment for creating a hydrophilic surface. For instance, the article can be exposed to a plasma treatment.

When formed into an implant, prior to being inserted into the body of a patient, the article can be sterilized. In one application, for instance, the molded implant can be subjected to a gamma sterilization treatment which can be carried out, for instance, at an energy level of from about 20 kGy to about 30 kGy. Alternatively, the implant can be sterilized using an E-beam sterilization process or a plasma sterilization process.

All different types of implants can be molded in accordance with the present disclosure. Implants that can be made according to the present disclosure, for instance, include joint replacements, spinal implants, craniofacial implants, orthopedic fixation devices, acetabular liners such as acetabular cups, finger joint prostheses, dental implants, and the like. Joint replacements can include hip implants, knee implants, and shoulder implants. For instance, the polymer composition of the present disclosure can be used to produce a femoral implant, a tibial insert including a tibial plateau, or a glenoid component in shoulder arthroplasty.

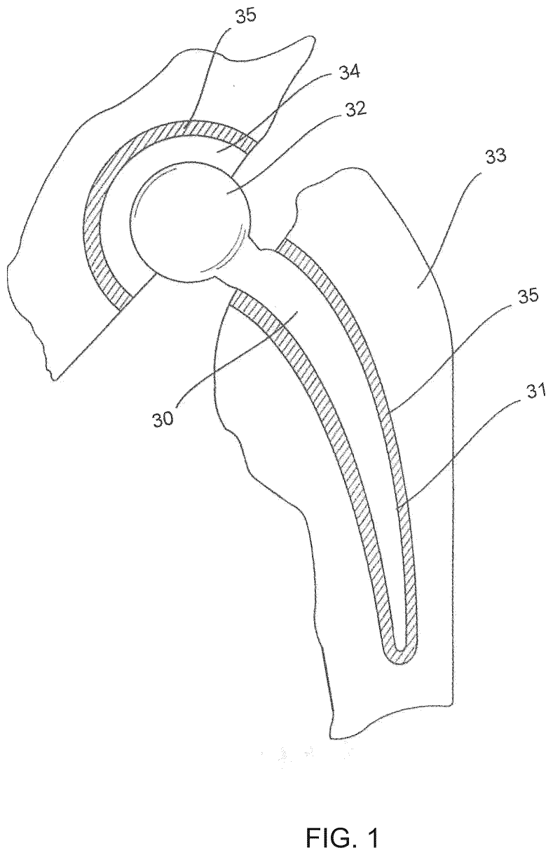

Referring to FIG. 1, for exemplary purposes only, a hip implant is illustrated that can include components made according to the present disclosure.

The hip prosthesis 30 can be made from a polymer material in accordance with the present disclosure. As shown, the hip prosthesis 30 has been inserted into a cavity reamed into a bone 33, such as a femur.

The hip implant illustrated in FIG. 1 further includes an acetabular cup 34 that can also be made in accordance with the present disclosure. Acetabular cup 34 comprises a joint engaging member that is adapted to receive the head 32 of the hip prosthesis 30. In order to implant the hip prosthesis 30 and the acetabular cup 34, both articles can be cemented to the bone using a bone cement 35.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only and is not intended to limit the invention so further described in such appended claims.

What is claimed:

1. A polymer composition for producing implants comprising:
an ultra-high molecular weight polyethylene in the form of particles, wherein the ultrahigh molecular weight polyethylene particles display a bulk density of greater than about 0.46 g/cm³, the ultra-high molecular weight polyethylene having an average molecular weight of from about 3,000,000 g/mol to about 12,000,000 g/mol, the ultra-high molecular weight polyethylene particles having an average particle size (D50) of from about 100 microns to about 200 microns, the polymer composition displaying an Izod impact strength of at least about 126 kJ/m² when tested according to ASTM F648-21, Annex A1, the polymer composition containing the ultra-high molecular weight polyethylene in an amount of about 99% by weight or greater, the ultra-high molecular weight polyethylene having a density of from about 927 to about 944 kg/m³ when tested according to ASTM D792 or D1505.

2. A polymer composition as defined in claim 1, wherein the ultra-high molecular weight polyethylene displays a bulk density of greater than about 0.48 g/cm³.

3. A polymer composition as defined in claim 1, wherein the ultra-high molecular weight polyethylene displays an average molecular weight of from about 3,800,000 g/mol to about 4,800,000 g/mol.

4. A polymer composition as defined in claim 1, wherein the ultra-high molecular weight polyethylene contains aluminum in an amount from about 10 ppm to about 35 ppm.

5. A polymer composition as defined in claim 1, wherein the ultra-high molecular weight polyethylene contains titanium in an amount less than about 20 ppm.

6. A polymer composition as defined in claim 1, wherein the ultra-high molecular weight polyethylene particles have an average particle size (D50) of from about 120 microns to about 160 microns.

7. A polymer composition as defined in claim 1, wherein the polymer composition is free of stabilizers, antioxidants, and processing aids.

8. A polymer composition as defined in claim 1, wherein the ultra-high molecular weight polyethylene comprises a polyethylene homopolymer according to ASTM D4020.

9. A polymer composition as defined in claim 1, wherein the polymer composition contains the ultra-high molecular weight polyethylene polymer in an amount of at least about 99.9% by weight.

10. A polymer composition as defined in claim 1, wherein the ultra-high molecular weight polyethylene displays a viscosity number of from about 2,000 ml/g to about 3,200 ml/g when tested according to ASTM D4020.

11. A polymer composition as defined in claim 1, wherein the ultra-high molecular weight polyethylene contains calcium in an amount of about 5 mg/kg or less.

12. A polymer composition as defined in claim 1, wherein the ultra-high molecular weight polyethylene contains chlorine in an amount of about 30 mg/kg or less.

13. A medical implant made from the polymer composition as defined in claim 1.

14. An implant as defined in claim 13, wherein the implant has been compression molded from the polymer composition.

15. An implant as defined in claim 13, wherein the implant has been ram extruded from the polymer composition.

16. An implant as defined in claim 13, wherein the implant comprises a hip prosthesis or a knee prosthesis.

17. An implant as defined in claim 13, wherein the implant comprises an acetabular cup.

18. An implant as defined in claim 13, wherein the implant comprises a tibial plateau.

19. A polymer composition for producing implants comprising:
an ultra-high molecular weight polyethylene in the form of particles, the ultra-high molecular weight polyethylene containing aluminum in an amount from about 10 ppm to about 50 ppm, the ultra-high molecular weight polyethylene having an average molecular weight of from about 3,000,000 g/mol to about 12,000,000 g/mol, the ultra-high molecular weight polyethylene particles having an average particle size (D50) of from about 100 microns to about 200 microns, the polymer composition displaying an Izod impact strength of at least about 126 kJ/m² when tested according to ASTM F648-21, Annex A1, the polymer composition containing the ultra-high molecular weight polyethylene in an amount of about 99% by weight or greater, the ultra-high molecular weight polyethylene having a density of from about 927 to about 944 kg/m³ when tested according to ASTM D792 or D1505.

20. A polymer composition as defined in claim 19, wherein the ultra-high molecular weight polyethylene displays a bulk density of greater than about 0.48 g/cm³.

21. A polymer composition as defined in claim 19, wherein the ultra-high molecular weight polyethylene contains aluminum in an amount from about 10 ppm to about 20 ppm.

22. A polymer composition as defined in claim 19, wherein the ultra-high molecular weight polyethylene contains titanium in an amount less than about 20 ppm.
